(19) United States
(12) Patent Application Publication
(10) Pub. No.: US 2025/0127461 A1
(43) Pub. Date: Apr. 24, 2025

(54) METHOD RELATING TO PULMONARY HYPERTENSION

(71) Applicants: Steffen Fuchs, Lahnau (DE); Alexander Zom, Heuchelheim (DE); JUSTUS-LIEBIG-UNIVERSITAET GIESSEN, Giessen (DE)

(72) Inventors: Steffen Fuchs, Lahnau (DE); Alexander Zorn, Heuchelheim (DE); Henning Gall, Heuchelheim (DE); Ardeschir Ghofrani, Wettenberg (DE); Manuel Richter, Giessen (DE); Werner Seeger, Biebertal (DE); Khodr Tello, Lich (DE); Athiththan Yogeswaran, Geissen (DE)

(21) Appl. No.: 18/919,199

(22) Filed: Oct. 17, 2024

(30) Foreign Application Priority Data
Oct. 20, 2023 (EU) 23204781.1

(51) Int. Cl. A61B 5/00; G16H 10/60; G16H 50/30

(52) U.S. Cl. CPC A61B 5/7275; A61B 5/7264; G16H 10/60; G16H 50/30

(57) ABSTRACT

The invention relates to a method for determining a score value (S) for the likelihood of the presence of a pulmonary hypertension (PH) in a person (P).

The invention relates to a method for determining a score value for the likelihood of the presence of a pulmonary hypertension (PH) with the aid of the end-tidal partial pressure of carbon dioxide (PₑₜCO₂).

### DESCRIPTION OF AND INTRODUCTION TO THE GENERAL FIELD OF THE INVENTION

The amount of blood flowing through the pulmonary circulation at any time is the same as that flowing through all the other organs of the body, namely about five liters per minute. At rest, this is about 7200 liters per day. Under exertion, this amount of blood can be increased many times over.

The healthy lung is constructed like an elastic sponge, and so the flow resistance in the pulmonary vessels is normally very low: pumping 5 L/min of cardiac output through the lungs requires only 14 mmHg (1866.51 Pa) of pressure—pumping the same amount through a straw would require about 5 times the pressure. The low pulmonary resistance means that, in direct comparison to the high-pressure system of the human body, the right side of the heart must normally build up only one sixth of the pressure of the system in order to pump the blood through the lungs.

Efficient gas exchange and hence supply of oxygen to all cells of the body requires exact cooperation of the lungs and heart. Any disruptions in this cooperation in the cardiopulmonary system can lead to pulmonary hypertension (PH). PH is therefore a pathophysiological phenomenon that leads clinically to symptoms such as dyspnea (shortness of breath). Experience from daily clinical practice shows that the classification of the various forms of PH is not always unambiguously possible, even for specialized physicians.

In view of the rising number of patients showing symptoms such as dyspnea and therefore being examined for pulmonary hypertension (PH) and in view of the considerable costs, the time required and the potential risk when assessing pulmonary vascular diseases, there is interest in developing novel, noninvasive diagnostic methods for identifying a pulmonary hypertension (PH) in patients without the risks due to an invasive diagnostic procedure.

### PRIOR ART

Currently, definitive confirmation of the diagnosis PH requires a right heart catheter (RHC). Although there are clinical and echocardiographic features that make a PH more likely, these indicators are often insufficient to dispense with an RHC in patients with elevated right ventricular systolic pressure (RVSP) in echocardiography. In addition, a normal RVSP in echocardiography is insufficient to rule out a PH. In very many cases, pulmonary hypertension is therefore unfortunately only diagnosed too late or not at all, with far-reaching consequences for the individual patient. To diagnose a PH, the current standard procedure is to first carry out an echocardiography. However, as shown by comparative studies, this is not always sufficiently reliable in terms of sensitivity, specificity and hence accuracy despite the effort, the limited availability and the necessary expertise.

### OBJECT

It is an object of the present invention to reduce the disadvantages of the prior art, such that use can be made of the measurement of the end-tidal partial pressure of CO₂ for diagnosing pulmonary hypertension. This allows a more precise and more reliable diagnosis that can be made for patients in a noninvasive and cost-effective manner.

### ACHIEVEMENT OF THE OBJECT

This object is achieved by the features of the main claim. Furthermore, advantageous embodiments and developments of the invention can be found in the dependent claims.

The terms relevant to the method according to the invention will be defined first of all:

### Measurement Data Concerning the End-Tidal Partial Pressure of CO₂

The value of the end-tidal partial pressure of CO₂ (EtCO2) is an important characteristic for allowing assessment of pulmonary function.

In particular, a particularly low value of the end-tidal partial pressure of CO₂ (EtCO2) is a strong indication that a pulmonary hypertension (PH) may be present.

What is measured is the percentage or partial pressure of carbon dioxide in capnometry. The normal range for the end-expiratory partial pressure of carbon dioxide is 33 to 43 mmHg or, if specifying the measurement value as a concentration, 4.3% to 5.7% by volume. The conversion between the two units depends on the ambient air pressure, which is therefore also measured by the instrument. The gold standard for measuring the partial pressure of CO₂ in the blood is arterial blood gas analysis. Because of its invasiveness, it usually only allows a snapshot and is therefore of only limited usability, especially for outside a hospital. Continuous measurement of the partial pressure of CO₂ in the blood can, however, be accomplished by measuring the partial pressure of CO₂ in the exhaled air of the patient. This is referred to as capnometry. The partial pressure of CO₂ is determined at the end of maximal exhalation, and so the term used is the end-tidal partial pressure of CO₂ (PₑₜCO₂). Another, less widespread method is transcutaneous capnometry, the measurement of the CO₂ value across the skin (P_{tc}CO₂). This method of transcutaneous measurement of CO₂ does not depict the CO₂ in exhaled air. It is therefore unsuitable for examining pulmonary diseases.

The various methods differ in their function and their results in relation to the actual paCO₂.

End-tidal capnography is the most widespread method for noninvasive measurement of the partial pressure of CO₂. Its main applications are usually within the scope of monitoring anesthesia or for diagnosing pulmonary vascular diseases.

Measurement can be made by means of infrared measurement in the mainstream method or in the sidestream method in relation to the ventilation circuit. In the mainstream method, the CO₂ level in the exhaled air of the patient is measured. In the sidestream method, a certain amount of gas is continuously withdrawn from the main stream by a small additional line and evaluated in the analyzer of the ventilator. Modern capnometers use the smallest possible amounts of gas for this purpose, which are usually between 30-50 ml/min.

### Dynamic Clinical Parameter P1:

Dynamic clinical parameters P1 can include either ECG measurement data or a combination of ECG measurement data and MRI measurement data.

ECG stands for electrocardiography and refers to an examination method in which the electrical activity of the heart is measured by means of electrodes on the body surface. Regular electrical impulses control the heartbeat. They are triggered by the sinus node in the right atrium of the heart and spread across the heart muscle as small impulses. This causes contraction of first the atria and then the ventricles. The spread of electrical stimuli in the heart muscle is also measurable on the skin. An ECG measures these electrical potential differences at different points on the body and displays them as curves. These ECG curves are called electrocardiograms.

If the heart is beating regularly, the typical ECG pattern is produced: The first deflection (P wave) shows how the electrical impulse (excitation) is spreading across the atria of the heart. The atria contract, pass blood into the ventricles and immediately return to a relaxed state. The excitation then reaches the ventricles. This is visible in the ECG as Q, R and S waves, known as the QRS complex, which is associated with the contraction of the ventricles. After that, the T wave indicates that the excitement is receding and the ventricles are returning to a relaxed state.

The chronologically ordered data of the sequence of excitation of the heart are referred to hereinafter as ECG (measurement) data for simplicity.

The method according to the invention for determining a score value S for the likelihood of the presence of a pulmonary hypertension in a person P from the end-tidal partial pressure of CO₂ comprises at least the following steps
I. providing the CO₂ measurement data concerning the end-tidal partial pressure of CO₂ (PₑₜCO₂) of the person (P) over a period (t)
In a first step, CO₂ measurement data D1 concerning the end-tidal partial pressure of CO₂ are provided. The end-tidal partial pressure of CO₂ is measured from the exhaled respiratory air outside the human body.
Measurement can be carried out, for example, by means of end-tidal capnography.
II. providing ECG measurement data of the person (P) as dynamic clinical parameter (P1) and determining at least the value of the Sokolow-Lyon index, the value of the S wave in V5, the value of the R wave in V4 and the value of the angle of the QRS axis from the ECG measurement data of said person (P) over the same period (t) as in step I
   Said ECG measurement data are the data available after completion of the ECG measurement on a person. The providing begins only after completion of the examination with data collection on the human body, i.e., only after the measurement on the patient has been completed.
III. determining a score value (S) for the likelihood of the presence of a pulmonary hypertension in said person from the CO₂ measurement data concerning the end-tidal partial pressure of CO₂ (PₑₜCO₂) and at least the ECG measurement data as dynamic clinical parameter (P1) in the following substeps
   a. comparing the PₑₜCO₂ from step I with a limit value G1,
      if PₑₜCO₂ is greater than or equal to G1, S is equal to 1,
      if PₑₜCO₂ is less than G1, the method is continued in step IIIb,
   b. comparing the value of the Sokolow-Lyon index from step II with a limit value G2,
      if Sokolow-Lyon index is greater than or equal to G2, S is equal to 1,
      if Sokolow-Lyon index<G2, the method is continued in step IIIc,
   c. comparing the value of the S wave in V5 from step II with a limit value G3,
      if S wave in V5 is greater than or equal to G3, S is equal to 1,
      if S wave in V5 is less than G3, the method is continued in step IIId,
   d. comparing the value of the R wave in V4 from step II with a limit value G4,
      if R wave in V2 is less than G4, S is equal to 1,
      if R wave in V2 is greater than or equal to G4, the method is continued in step IIIe,
   e. comparing the value of the angle of the QRS axis from step II with a limit value G5,
      if angle of the QRS axis is greater than G5, S is equal to 1,
      if angle of the QRS axis is less than or equal to G5, S is equal to 0.

The method is carried out by means of a device for electronic data processing: PC, smartphone, Mac©. It is a computer-implemented method.

In addition to clinical and ECG measurement data, what is determined in particular is the screening score recommended in the current guidelines for assessing the likelihood of the presence of a pulmonary hypertension. In combination with the end-tidal partial pressure of CO₂ (PₑₜCO₂), an improvement in the predictive power of each individual test can be achieved.

The score value is determined iteratively.

First, a classification is made on the basis of the PetCO₂.

A decision tree for classification into the groups “PH likely” and “PH unlikely” is first made on the basis of the measurement data of the end-tidal CO2 value (PetCO₂). The limit value G1 accepted by experts is about 31.5 mmHg. However, in practice, the PₑₜCO₂ value alone often does not have sufficient sensitivity and specificity to make definitive diagnostic statements. As explained above, additional parameters are also included in this limit value. This limit value is therefore applied in the application described, but non-dynamic parameters are then also included in the score in the manner described above.

If the measured PₑₜCO₂ is greater than or equal to G1, the score value is 1 and a PH is considered unlikely.

If the measured PₑₜCO₂ is less than or equal to G1, the diagnosis PH is considered initially likely (score value S equal to 0).

Thereafter, further parameters determined from the ECG measurement data (Sokolow-Lyon index, S wave in V5 (SV5), R wave in V2 (RV2) and angle of the QRS axis) and preferably parameters determined from MRI image data are used to carry out a further classification taking into account the pre-existing limit values.

A particularly advantageous parameter P1 is the Sokolow-Lyon index, which is obtained from the ECG measurement data.

The Sokolow-Lyon index is a nonspecific ECG sign to indicate the presence of left heart hypertrophy or right heart hypertrophy. The Sokolow index is calculated by using Wilson's chest leads. A limit value G2 for the Sokolow-Lyon index currently accepted by experts is 0.5.

If the measured Sokolow-Lyon index is greater than or equal to G2, the score value is equal to 1 and a PH is considered unlikely.

This is accomplished by adding the initially highest amplitude (in mV) of the S wave in lead V1 or V2 and of the R wave in V5 or V6. If the sum total of the two amplitudes exceeds 3.5 mV (35.0 mm), this indicates a PH.

The calculation of the Sokolow index for RVH is also carried out using V1 or V2 and V5 or V6, except that the respective other wave is used. The deflection of the R wave in V1 or V2 is added to the deflection of the S wave in V5 or V6. If the sum total of the two amplitudes is greater than 1.05 mV (10.5 mm), this indicates the likely presence of a PH.

However, the Sokolow-Lyon index alone has insufficient sensitivity and specificity to make definitive diagnostic statements. It is therefore combined at least with the measurement data for the end-tidal partial pressure of CO₂ (PₑₜCO₂) for determination of the score value S. However, as a nonspecific ECG sign, the Sokolow-Lyon index can contribute to formulating substantiated suspected diagnoses.

If the Sokolow-Lyon index is less than G2, the method is continued and the S wave in V5 is noted and compared with a limit value G3. A common limit value G3 for the S wave in V5 is 0.575 mV. If the S wave in V5 is greater than or equal to G3, then a PH is again considered unlikely and S is equal to 1. If this is not the case, the method is continued and the R wave in V2 is noted and compared with a limit value G4. A common limit value G4 on the R wave in V2 is 0.125 mV. If the R wave in V2 is less than G4, then S is equal to 1. If this is not the case, the method is continued and the QRS axis is noted and its angle is compared with a limit value G5. A common limit value G5 for the angle of the QRS axis is 65°. If the angle of the QRS axis is less than G5, S is equal to 1; if the angle of the QRS axis is less than or equal to 65°, S is equal to 0.

The limit values G1, G2, G3, G4 and G5 for PₑₜCO₂, Sokolow-Lyon index, SV5, RV2, and the angle of the QRS axis correspond to the currently common limit values for patients with no previous diseases and were determined from comparative studies.

This score value S for the presence of a pulmonary hypertension is an interim result relevant to diagnosis and must still be interpreted by medical professionals for a diagnosis in a medical context. For example, the threshold for the presence of a pulmonary hypertension may be a score value S of 0.5. Values of 0.5 or less are identified as pulmonary hypertension. This threshold has been determined by medical professionals on the basis of experience by way of example and is not part of the method according to the invention.

When evaluating the measurement data, the likelihood of the presence of a pulmonary hypertension or the severity thereof is critical. The interpretation of the score value S to make a specific diagnosis is reserved for medical professionals and is not part of the method. A typical threshold for the presence of a pulmonary hypertension may be 0.5 for example; that is to say, values of 0.5 or less are identified as a high likelihood of the presence of a (relevant) pulmonary hypertension.

The combination of measurement data concerning the end-tidal partial pressure of CO₂ with measurement data of at least one further parameter P1 of the cardiovascular system over the same period t leads to a much safer and more reliable method of diagnosing a pulmonary hypertension that can be carried out noninvasively on a patient.

The method according to the invention also allows the classification of the likelihood of pulmonary hypertension from the end-tidal partial pressure of CO₂ in exhaled respiratory air.

In the method according to the invention, a widely available diagnostic method (measurement of the end-tidal partial pressure of CO₂) is used as a basis to predict the result of a diagnostic method of only limited availability (e.g., right heart catheter), thus expanding the diagnostic possibilities of measurement of end-tidal CO₂.

### Exemplary Embodiment

As an illustrated example, FIG. 2 shows the usefulness of the end-tidal partial pressure of CO₂ for the detection of a precapillary PH (PAH or chronic thromboembolic PH). The diagnosis “PH” or “No PH” was verified invasively by means of a right heart catheter in comparative tests:

The value of the end-tidal partial pressure of CO₂ per se shows a sensitivity of 84% at the optimal cut-off of 31.5 mmHg determined according to Youden. The Youden method is an index used as a measure of an optimal limit value in receiver operating characteristic curve analyses. However, the specificity is greatly limited; it is only 51%:

The addition of ECG measurement data such as the Sokolow-Lyon index and the height of the R wave or S wave in various leads increases both the sensitivity and specificity of the detection method (sensitivity: 86%; specificity: 68%). Here, as already mentioned above, classification is carried out on the basis of PₑₜCO₂. Subsequently, for example in patients with high PₑₜCO₂, a positive Sokolow-Lyon index is considered to be a sign of right heart strain, and so “PH unlikely” is reclassified as “PH likely”. This shows that it is advantageous to combine the measurement value PₑₜCO₂ with other parameters.

The exemplary embodiment shown is merely an example of the usefulness of a combination of PₑₜCO₂ with a further parameter P1 in a preliminary test cohort and is therefore to be understood as an example procedure.

In an example case, a patient X has a PₑₜCO₂ of 30.7 mmHg. This value is therefore less than the limit value G1 of 31.5 mmHg. A PH is therefore initially likely. For further differentiation, the Sokolow-Lyon index was then determined. This is less than 0.5. Therefore, PH remains likely. With the next step, the SV5 value was determined. This is 0.3, meaning that pulmonary hypertension appears to remain likely. RV2 was then determined. This is 0.2, meaning that a pulmonary hypertension is still likely. Finally, the angle of the QRS axis of the patient was determined. This is 61° and is thus less than the limit value G5 of 65°, and there is therefore no indication for PH and thus a low likelihood of PH, meaning that S is equal to 1.

The prediction that there is no pulmonary hypertension in this patient was confirmed by a complementary diagnosis by means of a right heart catheter.

### FIGURES

FIG. 1 shows the algorithm for determination of the score value. The limit values in this example correspond to the currently common limit values for patients with no previous diseases.

FIG. 2 shows the selectivity and sensitivity in determining detection of a PH.

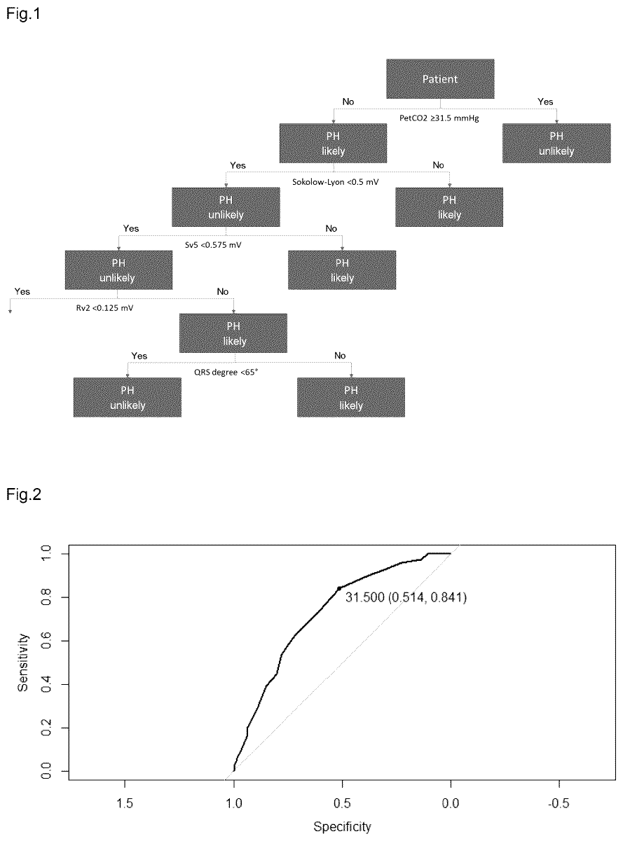

1. A method for determining a score value (S) for the likelihood of the presence of a pulmonary hypertension (PH) in a person (P), comprising at least the following steps:
I. providing the CO₂ measurement data concerning the end-tidal partial pressure of CO₂ (PₑₜCO₂) of the person (P) over a period (t);
II. providing ECG measurement data of the person (P) and determining at least the value of the Sokolow-Lyon index, the value of the S wave in V5, the value of the R wave in V4 and the value of the angle of the QRS axis from the ECG measurement data of said person (P) as dynamic clinical parameter (P1) over the same period (t) as in step I; and
III. determining a score value (S) for the likelihood of the presence of a pulmonary hypertension in said person from the CO₂ measurement data concerning the end-tidal partial pressure of CO₂ (PetCO₂) from step I and at least the ECG measurement data as dynamic clinical parameters (P1) from step II in the following substeps, where S equal to 0 means a high likelihood of a pulmonary hypertension (PH) and S equal to 1 means a low likelihood of a pulmonary hypertension (PH);
a. comparing the PetCO₂ from step I with a limit value G1,
if PₑtCO₂ is greater than or equal to G1, S is equal to 1,
if PₑₜCO₂ is less than G1, the method is continued in step IIIb,
b. comparing the value of the Sokolow-Lyon index from step II with a limit value G2,
if Sokolow-Lyon index is greater than or equal to G2, S is equal to 1,
if Sokolow-Lyon index<G2, the method is continued in step IIIc,
c. comparing the value of the S wave in V5 from step II with a limit value G3,
if S wave in V5 is greater than or equal to G3, S is equal to 1,
if S wave in V5<G3, the method is continued in step IIId,
d. comparing the value of the R wave in V4 from step II with a limit value G4,
if R wave in V2 is less than G4, S is equal to 1,
if R wave in V2 is greater than or equal to G4, the method is continued in step IIIe,
e. comparing the value of the angle of the QRS axis from step II with a limit value G5,
if angle of the QRS axis is less than G5, S is equal to 1,
if angle of the QRS axis is less than or equal to G5, S is equal to 0.
